(19) United States
(12) Patent Application Publication
(10) Pub. No.: US 2023/0181052 A1
(43) Pub. Date: Jun. 15, 2023

(54) BIOLOGICAL INFORMATION ACQUISITION DEVICE, BIOLOGICAL INFORMATION ACQUISITION METHOD, AND RECORDING MEDIUM

(71) Applicant: NEC Corporation, Minato-ku, Tokyo (JP)

(72) Inventors: Kazuki IHARA, Tokyo (JP); Hiroyuki ENDOH, Tokyo (JP)

(73) Assignee: NEC Corporation, Minato-ku, Tokyo (JP)

(21) Appl. No.: 18/015,279

(22) PCT Filed: Jul. 15, 2020
(86) PCT No.: PCT/JP2020/027456

§ 371 Date: Jan. 9, 2023

(51) Int. Cl. A61B 5/026; A61B 5/024; A61B 5/00

(52) U.S. Cl. CPC A61B 5/026; A61B 5/024; A61B 5/7235

(57) ABSTRACT

According to the present invention, in a configuration in which a biological signal is received from an attachment-type device including one or more light emitting means and two or more light receiving means, information on a living body is acquired with high accuracy. A modulation unit (110) dims the one or more light emitting means with a specific frequency, a receiving unit (120) receives a biological signal based on the light received from a living body through the two or more light receiving means, and an adjusting unit (130) adjusts the intensity of the biological signal acquired from each light receiving means on the basis of the component of a specific frequency of the biological signal.

### TECHNICAL FIELD

The present invention relates to a biological information acquisition device, a biological information acquisition method, and a recording medium, and particularly relates to a biological information acquisition device, a biological information acquisition method, and a recording medium for acquiring information regarding a living body using light.

### BACKGROUND ART

There has been a biological information acquisition device that acquires information regarding a living body using a set of one light emitting unit and one light receiving unit. The biological information acquisition device may also be called a running watch, a fitness tracker, or an activity meter depending on its purpose of use. PTL 1 describes an example of a related art.

The information regarding the living body is, for example, an index value relating to a mental and physical health condition such as a heart rate, a blood flow rate, or a blood oxygen concentration. A method for detecting biological information reflecting activity of an autonomic nervous system such as changes in pulse has been widely known. Furthermore, it has become possible to estimate an emotion of a living body through a model from limited biological information such as changes in pulse (e.g., PTL 1).

Many so-called smart devices capable of cooperating with health management applications, life log functions, and the like of smartphones have also been developed. Examples of smart devices include a wristband type device to be worn on an arm and an anklet type device to be worn on an ankle. Furthermore, there has also emerged a stick-on type device having a flexible substrate provided with a light emitting means and a light receiving means to be stuck onto a skin of a living body (e.g., PTL 2).

In addition, there has been developed a stick-on type sensor device in which sensors including one or more light emitting means and two or more light receiving means are constructed in an array form. Some of such stick-on type sensor devices are called super bio imagers. By using biosignals acquired from respective light receiving means constituting the sensors in the array form, the super bioimager can perform more advanced processing (e.g., statistical calculation) than a stick-on type device including a single sensor. Therefore, the super bioimager can acquire biological information such as pulse more accurately (NPL 1). Note that not only the super bioimagers but also any sensor device in which sensors including one or more light emitting means and two or more light receiving means are constructed in an array form can have the above-described advantages as compared with a stick-on type device including a single sensor.

### CITATION LIST

### Patent Literature

[PTL 1] JP 2018-504188 A
[PTL 2] JP 2011-50745 A
[PTL 3] WO 2018/167854 A

### Non Patent Literature

[NPL 1] Yokota, T., Nakamura, T., Kato, H. et al. “A conformable imager for biometric authentication and vital sign measurement.”, Nat Electron (2020).

### SUMMARY OF INVENTION

### Technical Problem

The sensor device is twisted as a living body moves. In addition, each user may mount the sensor device on his/her living body in a different way. In the sensor device in which sensors including one or more light emitting means and two or more light receiving means are constructed in an array form, since the light receiving means are different from each other in a magnitude of noise generated therein depending on how the sensor device is stuck onto the living body and how the sensor device is twisted as the body moves, it is difficult to completely remove only noises, such as artifacts as the body moves, from biosignals. This makes it difficult to accurately acquire information regarding the living body from the biosignals.

The present invention has been made in view of the above-described problem, and an object of the present invention is to acquire information regarding a living body with high accuracy in a configuration for receiving a biosignal from a stick-on type device including one or more light emitting means and two or more light receiving means.

### Solution to Problem

According to an aspect of the present invention, a biological information acquisition device includes: a modulation means configured to dim one or more light emitting means at a specific frequency; a receiving means configured to receive biosignals based on light received from a living body by two or more light receiving means; and an adjustment means configured to adjust an intensity of the biosignal acquired from each of the light receiving means based on a specific frequency component for the biosignal.

According to another aspect of the present invention, a biological information acquisition method includes: dimming one or more light emitting means at a specific frequency; receiving biosignals based on light received from a living body by two or more light receiving means; and adjusting an intensity of the biosignal acquired from each of the light receiving means based on a specific frequency component for the biosignal.

According to another aspect of the present invention, a recording medium stores a program for causing a computer to execute: dimming one or more light emitting means at a specific frequency; receiving biosignals based on light received from a living body by two or more light receiving means; and adjusting an intensity of the biosignal acquired from each of the light receiving means based on a specific frequency component for the biosignal.

### Advantageous Effects of Invention

According to an aspect of the present invention, it is possible to acquire information regarding a living body with high accuracy in a configuration for receiving a biosignal from a stick-on type device including one or more light emitting means and two or more light receiving means.

### BRIEF DESCRIPTION OF DRAWINGS

FIG. 1 schematically illustrates a configuration of a system common to all example embodiments.

FIG. 2 is a block diagram illustrating an example of a configuration of a stick-on type device included in a system common to all example embodiments.

FIG. 3 is a diagram illustrating how an exemplary stick-on type device included in a system common to all example embodiments looks in a plan view.

FIG. 4 is a plan view illustrating a modification of a stick-on type device included in a system common to all example embodiments.

FIG. 5 is a cross-sectional view illustrating a configuration of the stick-on type device illustrated in FIG. 4.

FIG. 6 is a block diagram illustrating an example of a configuration of a biological information acquisition device according to a first example embodiment.

FIG. 7 illustrates an example of a modulated signal generated by a modulation unit of the biological information acquisition device according to the first example embodiment.

FIG. 8 is a graph illustrating an example of a biosignal received by a receiving unit before a control signal is modulated by the modulation unit of the biological information acquisition device according to the first example embodiment.

FIG. 9 is a graph illustrating an example of a biosignal received by a receiving unit after a control signal is modulated by the modulation unit of the biological information acquisition device according to the first example embodiment.

FIG. 10 is a graph illustrating an example of a power spectrum obtained by Fourier transforming a biosignal.

FIG. 11 is a graph illustrating an example of a biosignal leveled by an adjustment unit of the biological information acquisition device according to the first example embodiment.

FIG. 12 is a cross-sectional view illustrating a first modification of the configuration of the stick-on type device according to the first example embodiment.

FIG. 13 is a cross-sectional view illustrating a second modification of the configuration of the stick-on type device according to the first example embodiment.

FIG. 14 is a cross-sectional view illustrating a third modification of the configuration of the stick-on type device according to the first example embodiment.

FIG. 15 is a cross-sectional view illustrating a fourth modification of the configuration of the stick-on type device according to the first example embodiment.

FIG. 16 is a block diagram illustrating a configuration of a biological information acquisition device according to a second example embodiment.

FIG. 17 is a block diagram illustrating a configuration of a biological information acquisition device according to a third example embodiment.

FIG. 18 is a block diagram illustrating a configuration of a biological information acquisition device according to a fourth example embodiment.

FIG. 19 is a diagram illustrating a hardware configuration of the biological information acquisition device according to any one of the first to fourth example embodiments.

### EXAMPLE EMBODIMENTS

Hereinafter, some example embodiments will be described. However, before describing some example embodiments, a configuration common to all the example embodiments will be described.

### Common to All Example Embodiments

### System 1

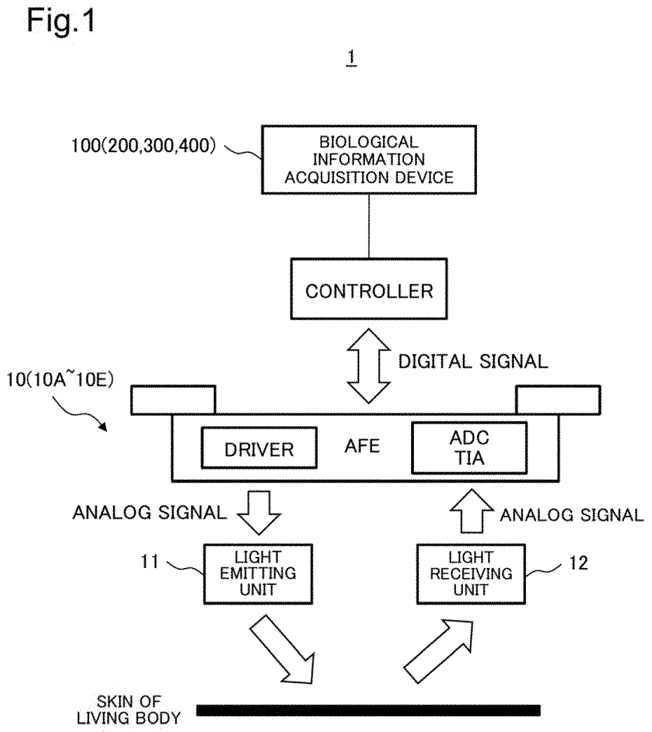

A configuration common to all example embodiments to be described below will be described with reference to FIGS. 1 to 3.

FIG. 1 schematically illustrates a configuration of a system 1 that can be commonly applied to all example embodiments. As illustrated in FIG. 1, the system 1 includes a biological information acquisition device 100 (200, 300, 400) and a stick-on type device 10. The biological information acquisition device 100 is a biological information acquisition device according to a first example embodiment, and also represents a minimum configuration for the biological information acquisition devices according to all the example embodiments.

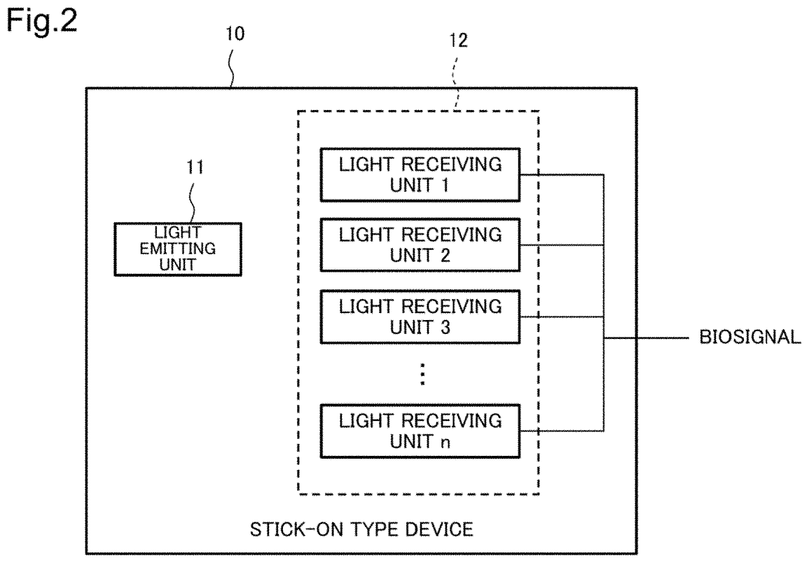

FIG. 2 is a block diagram illustrating an example of a configuration of the stick-on type device 10. In an example, the stick-on type device 10 is stuck onto a skin of a living body (such as a human body). As illustrated in FIG. 2, the stick-on type device 10 includes one or more light emitting units 11 and two or more light receiving units 12. In FIG. 2, n light receiving units 12 (n is an integer of 2 or more) are illustrated. The one or more light emitting units 11 are controlled by a driver of an analog front end (AFE). The one or more light emitting units 11 emit light in a specific wavelength range (e.g., visible light or near-infrared light) toward the skin of the living body.

In a case where the stick-on type device 10 includes a plurality of light emitting units 11, the plurality of light emitting units 11 may emit light having the same wavelength or may emit light in different wavelength ranges.

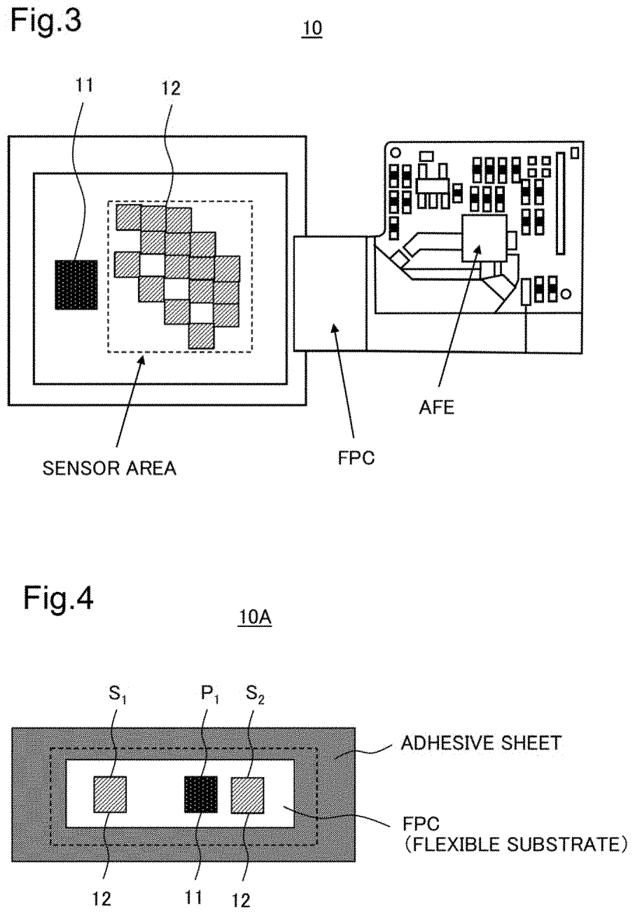

FIG. 3 is a plan view of the stick-on type device 10. As illustrated in FIG. 3, the AFE, the light emitting unit 11, and the light receiving units 12 are arranged on flexible printed circuits (FPCs). Note that the number of light receiving units 12 and the arrangement of the light receiving units 12 on a sensor area and a positional relationship between the light emitting unit 11 and the light receiving units 12 in FIG. 3 are all merely exemplary.

Some of the light emitted from the one or more light emitting units 11 is reflected by the skin of the living body, and the other is transmitted through the skin of the living body. The light transmitted through the skin of the living body is partially absorbed or scattered by various elements (e.g., hemoglobin in blood) constituting a tissue in the living body, and then released to the outside of the living body after passing through the skin of the living body again.

Each of the two or more light receiving units 12 included in the stick-on type device 10 receives light from the living body. Each of the two or more light receiving units 12 outputs an analog biosignal based on the light received from the living body to an A/D converter of the AFE. The analog biosignal is converted into a digital biosignal by the A/D converter, and then transmitted to a controller of the stick-on type device 10.

The controller of the stick-on type device 10 transmits a biosignal based on the light received from the living body to the biological information acquisition device 100 (200, 300, 400) by communicating with the biological information acquisition device 100 (200, 300, 400) in a wireless or wired manner.

The biological information acquisition device 100 (200, 300, 400) receives the biosignal based on the light from the living body from the controller of the stick-on type device 10. Then, the biological information acquisition device 100 (200, 300, 400) acquires information regarding the living body from the received biosignal. The information regarding the living body is, for example, an index value relating to a mental and physical health condition such as a heart rate, a blood flow rate, or a blood oxygen concentration. A configuration and an operation of the biological information acquisition device 100 (200, 300, 400) will be described in detail in each embodiment to be described below.

### Modification

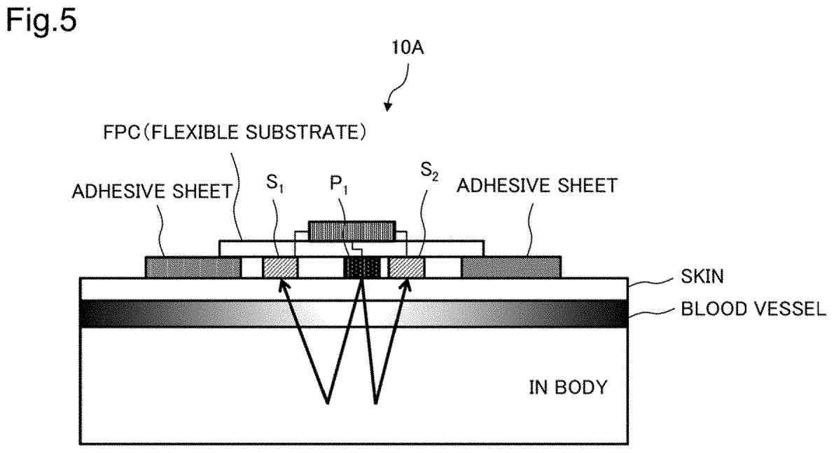

A modification of the stick-on type device 10 illustrated in FIG. 3 will be described with reference to FIGS. 4 and 5.

### Stick-On Type Device 10A

FIG. 4 is a plan view illustrating an example of a stick-on type device 10A according to a modification. As illustrated in FIG. 4, the stick-on type device 10A includes one light emitting unit 11 (denoted by reference sign P₁) and two light receiving units 12 (denoted by reference signs S₁ and S₂). The light emitting unit 11 and the light receiving units 12 are arranged on an FPC (also referred to as a flexible substrate). An end portion of a surface of the flexible substrate is bonded to an adhesive sheet having adhesiveness on both surfaces thereof. In the plan view illustrated in FIG. 4, a rectangular hole is formed in a central portion of the adhesive sheet, and the light emitting unit 11 and the light receiving unit 12 are disposed in the hole of the adhesive sheet.

FIG. 5 is a cross-sectional view of a stick-on type device 10A stuck onto a skin of a living body (a human body in an example). As illustrated in FIG. 5, the stick-on type device 10A is stuck onto the skin of the living body by the adhesive sheet with one light emitting unit 11 and two light receiving units 12 facing the living body. The light emitting unit 11 emits light toward the living body. Each of the two light receiving units 12 receives light from the living body.

Each of the two light receiving units 12 transmits a biosignal based on the received light in a wireless or wired manner to a biological information acquisition device 100 according to a first example embodiment to be described below.

### First Example Embodiment

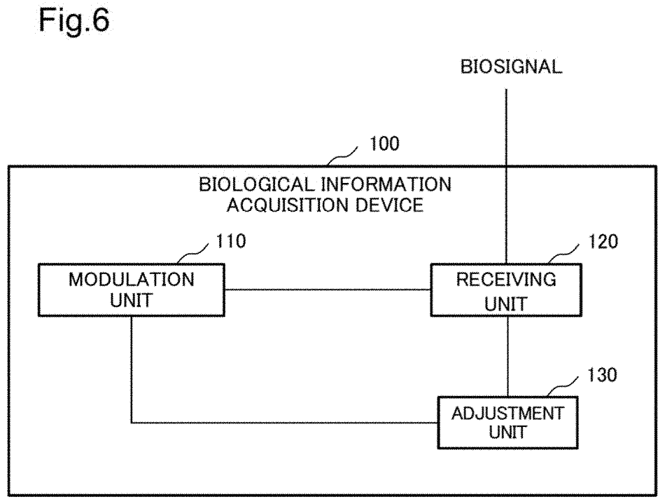

A first example embodiment will be described with reference to FIGS. 6 to 8.

### Biological Information Acquisition Device 100

FIG. 6 is a block diagram illustrating a configuration of a biological information acquisition device 100 according to the first example embodiment. As illustrated in FIG. 6, the biological information acquisition device 100 includes a modulation unit 110, a receiving unit 120, and an adjustment unit 130.

The modulation unit 110 dims one or more light emitting units 11 (an example of a light emitting means) at a specific frequency. The modulation unit 110 is an example of a modulation means. Here, the dimming refers to periodically changing an intensity of light emission from the one or more light emitting units 11. In an example, a control signal for controlling the one or more light emitting units 11 is output from a driver (FIG. 1) of the stick-on type device 10 or the stick-on type device 10A (hereinafter referred to as “the stick-on type device 10 (10A)”). The one or more light emitting units 11 are turned on (ON) or turned off (OFF) according to the control signal. In another example, the modulation unit 110 periodically changes a brightness of the one or more light emitting units 11 instead of switching ON and OFF (that is, blinking) the one or more light emitting units 11.

When notified from the receiving unit 120 that the biosignal has been received, the modulation unit 110 modulates the control signal for controlling the one or more light emitting units 11 at a specific frequency. Here, the specific frequency is preset according to what environment the stick-on type device 10 (10A) is used in or who uses the stick-on type device 10 (10A). An example of a method for determining a specific frequency will be described below.

The modulation unit 110 instructs the driver of the stick-on type device 10 (10A) to modulate the control signal at the specific frequency. The driver of the stick-on type device 10 (10A) outputs a control signal modulated at the specific frequency (hereinafter referred to as a modulated signal) to the one or more light emitting units 11.

The modulation unit 110 may dim the plurality of light emitting units 11 that emit light at the same wavelength at the same frequency. Alternatively, the modulation unit 110 may dim the plurality of light emitting units 11 that emit light at different wavelengths at different frequencies. Alternatively, the modulation unit 110 may dim the plurality of light emitting units 11 that emit light at the same wavelength at different frequencies.

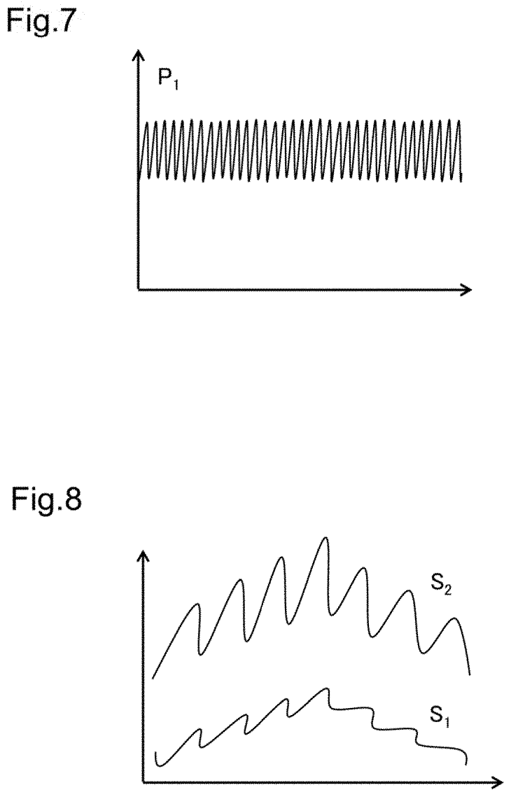

FIG. 7 illustrates an example of a modulated signal output from the modulation unit 110. In a graph illustrated in FIG. 7, the horizontal axis represents time, and the vertical axis represents voltage. In FIG. 7, the specific frequency is denoted by reference sign P₁. The one or more light emitting units 11 are alternately switched ON whenever an upper end of each wave in the modulated signal illustrated in FIG. 7 is reached and OFF whenever a lower end of each wave in the modulated signal.

In this manner, the modulation unit 110 modulates the control signal output from the driver of the stick-on type device 10 (10A). That is, the modulation unit 110 switches on and off the one or more light emitting units 11 every cycle corresponding to the specific frequency P₁, that is, every 1/P₁.

The modulation unit 110 notifies the adjustment unit 130 that the control signal is being modulated.

The receiving unit 120 receives biosignals based on light received from the living body by the two or more light receiving units 12 (an example of a light receiving means). The receiving unit 120 is an example of a receiving means. In an example, the receiving unit 120 receives two biosignals from the two light receiving units 12 of the stick-on type device 10 (10A) described above. The receiving unit 120 notifies the modulation unit 110 that the biosignals have been received from the light receiving units 12.

FIG. 8 illustrates examples of two biosignals (S₁, S₂) received by the receiving unit 120 from the two light receiving units 12 before the modulation unit 110 modulates a control signal.

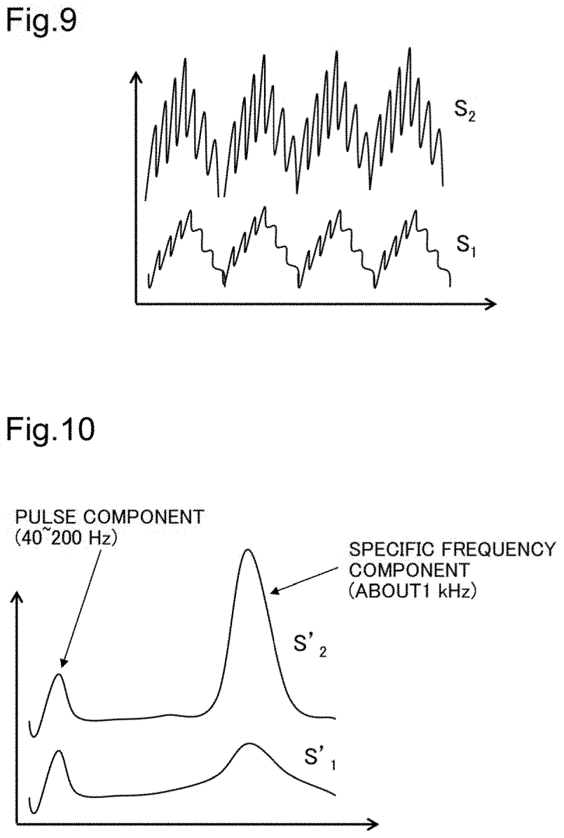

On the other hand, FIG. 9 illustrates examples of two biosignals (S₁, S₂) received by the receiving unit 120 from the two light receiving units 12 after the modulation unit 110 modulates the control signal. As illustrated in FIG. 9, changes based on the frequency P₁ of the modulated signal (FIG. 7) are superimposed on the biosignals (S₁, S₂) illustrated in FIG. 8. The receiving unit 120 outputs the two biosignals (S₁, S₂) illustrated in FIG. 9 to the adjustment unit 130.

The adjustment unit 130 adjusts an intensity of the biosignal acquired from each of the light receiving units 12, based on a specific frequency component for the biosignal. The adjustment unit 130 is an example of an adjustment means.

In an example, the adjustment unit 130 receives two biosignals (S₁, S₂) from the receiving unit 120. The adjustment unit 130 shifts the two biosignals (S₁, S₂) from a time domain to a frequency domain by Fourier transform.

FIG. 10 is a graph illustrating examples of power spectra (S′₁, S′₂) obtained by Fourier transforming the two biosignals (S₁, S₂) illustrated in FIG. 9, respectively. In each of the two power spectra (S′₁, S′₂) illustrated in FIG. 9, a frequency component (hereinafter referred to as a heart rate component) based on a heart rate of a living body exists in a range of about 40 to 200 Hz. In addition, in each of the two power spectra (S′₁, S′₂) illustrated in FIG. 9, a specific frequency component exists at about 1 kHz. The specific frequency components in the two power spectra (S′₁, S′₂) correspond to the frequency P₁ of the modulated signal (FIG. 7). That is, the specific frequency components in the two power spectra (S′₁, S′₂) are generated when the modulation unit 110 modulates the control signal of the driver.

The adjustment unit 130 levels the two biosignals (S₁, S₂) based on magnitudes of the specific frequency components in the two power spectra S′₂). More specifically, the adjustment unit 130 divides the intensity of the biosignal (S₁) by the magnitude of the specific frequency component in one power spectrum (S′₁). Similarly, the adjustment unit 130 divides the intensity of the biosignal (S₂) by the magnitude of the specific frequency component in the other power spectrum (S′₂).

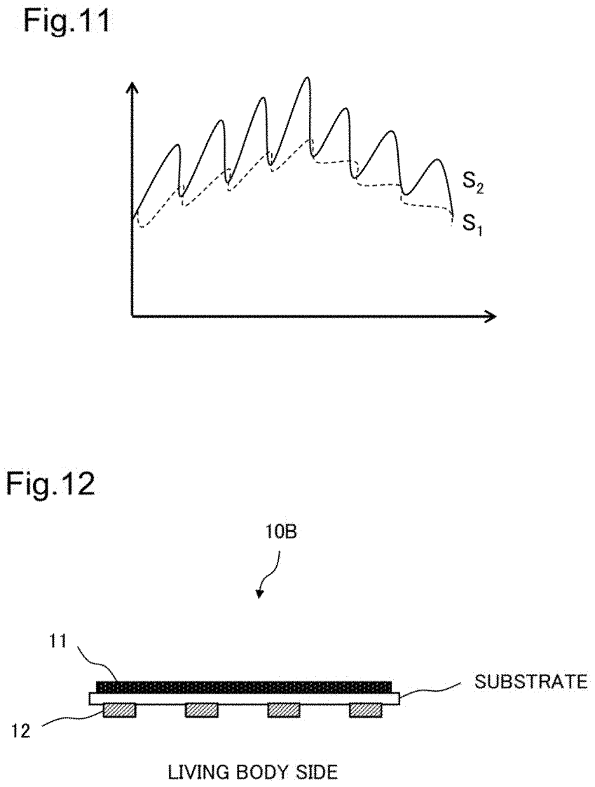

FIG. 11 illustrates two biosignals (S₁, S₂) after their intensities are adjusted by the adjustment unit 130. The adjustment unit 130 outputs the two biosignals (S₁, S₂) (FIG. 11) after their intensities are adjusted.

Both the stick-on type device 10 and the stick-on type device 10A (FIG. 4) described above are merely examples. Several modifications different from the stick-on type device 10 (10A) will be described below.

### First Modification

FIG. 12 illustrates an example of a stick-on type device 10B according to a first modification. In the stick-on type device 10B according to the first modification, one light emitting unit 11 and two or more light receiving units 12 are arranged on opposite surfaces of a transparent (or translucent) substrate (corresponding to the FPC in FIG. 4). The light emitting unit 11 is provided on an entire back surface (that is, a surface not facing the living body) of the substrate. In an example, the light emitting unit 11 is formed by arranging a plurality of light sources (such as chip light emitting diodes (LEDs)) at square lattice points on a printed board (different from the substrate illustrated in FIG. 12) having a mirror surface, and surrounding the light sources with a frame made of a plate whose inner surface is mirror-coated. By doing so, the light from the plurality of light sources is emitted toward the living body after being diffusely reflected by the mirror surface, thereby achieving plane emission. The light emitted from the one light emitting unit 11 penetrates through the transparent or translucent substrate and to be irradiated to the living body. However, in the first modification, in a case where a plurality of light receiving units 12 are provided as illustrated in FIG. 12, the light receiving units 12 are spaced apart from each other sufficiently, so that the light emitted from the one light emitting unit 11 passes between the light receiving units 12 toward the living body.

According to the configuration of the first modification, a wide range of the living body can be irradiated with light by the one light emitting unit 11, and accordingly, the biological information acquisition device 100 can acquire information regarding the living body from the wide range of the living body.

### Second Modification

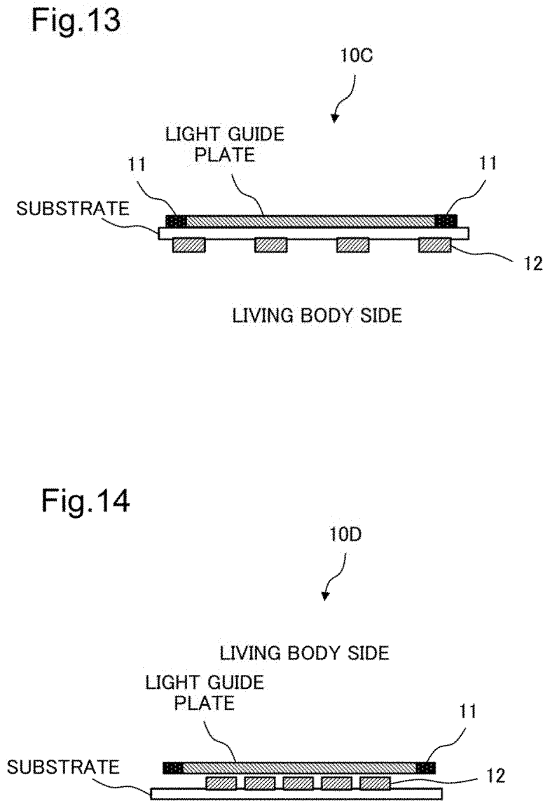

FIG. 13 illustrates an example of a stick-on type device 10C according to a second modification. In the stick-on type device 10C according to the second modification, similarly to the stick-on type device 10B according to the first modification, two light emitting units 11 and a large number of light receiving units 12 are arranged on opposite surfaces of a transparent (or translucent) substrate (corresponding to the FPC in FIG. 4). Furthermore, in the stick-on type device 10C according to the second modification, a light guide plate is disposed between the two light emitting units 11. Light emitted from the two light emitting units 11 is diffused in the light guide plate. In addition, a fine structure is provided on an inner surface (a surface facing the living body) of the light guide plate by inkjet printing or laser processing. Some of the light is scattered by the structure and emitted from the inside of the light guide plate toward a living body. According to the configuration of the second modification, it is possible to obtain an effect equivalent to that of the configuration (FIG. 12) of the first modification in which the large number of light receiving units 12 are provided on the entire back surface of the substrate. However, in the second modification, in a case where a plurality of light receiving units 12 are provided as illustrated in FIG. 13, the light receiving units 12 are spaced apart from each other sufficiently, so that the light emitted from the one or more light emitting units 11 passes between the light receiving units 12 toward the living body.

### Third Modification

FIG. 14 illustrates an example of a stick-on type device 10D according to a third modification. In the stick-on type device 10D according to the third modification, two light emitting units 11 and a light guide plate are provided on the same surface of the substrate as a large number of light receiving units 12. More specifically, in the stick-on type device 10D according to the third modification, the large number of light receiving units 12 and the two light emitting units 11 are laminated in this order on the surface of the transparent or translucent substrate. In an example, the light guide plate has a pinhole at a position corresponding to each light receiving unit 12, and a light shielding member is provided on a peripheral surface of each pinhole so that light does not leak from the inside of the light guide plate through the pinhole. As a result, the large number of light receiving units 12 can receive light from a living body through the pinholes. Alternatively, in a case where the light guide plate is transparent or translucent, some of the light from the living body toward the light guide plate penetrates through the light guide plate, without passing through the pinholes, to enter each of the two light receiving units 12. The light incident on an end portion of the light guide plate from the two light emitting units 11 is totally reflected (a so-called waveguide mode is established) by a difference in refractive index between the inside and the outside of the light guide plate, and thus is diffused to the entire inside of the light guide plate even if the light guide plate is transparent or translucent.

According to the configuration of the third modification, the two light emitting units 11 and the large number of light receiving units 12 can be integrally provided on the same surface of the substrate. The two light emitting units 11 can irradiate the living body with light via the light guide plate. In addition, the large number of light receiving units 12 can receive light from the living body via pinholes of the light guide plate or through the transparent or translucent light guide plate.

### Fourth Modification

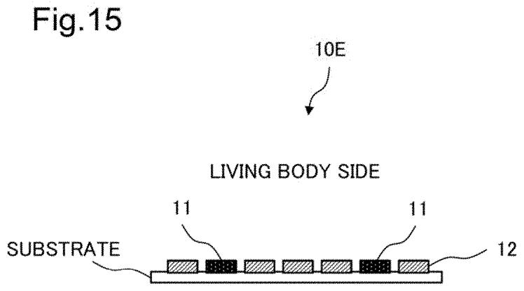

FIG. 15 illustrates an example of a stick-on type device 10E according to a fourth modification. In the stick-on type device 10E according to the fourth modification, two light emitting units 11 and a large number of light receiving units 12 are provided on a surface of a substrate. In addition, in the stick-on type device 10E according to the fourth modification, the two light emitting units 11 are spaced apart from each other, each being disposed between the light receiving units 12. It is preferable that light shielding members are provided between each of the light emitting units 11 and the light receiving units 12 on both sides of each of the light emitting units 11. This makes it possible to prevent light from the light emitting units 11 from directly entering the light receiving units 12.

According to the configuration of the fourth modification, similarly to the configuration of the third modification, the two light emitting units 11 can irradiate a living body with light without passing through the substrate. Furthermore, since the two light emitting units 11 are not covered by the light receiving units 12, the two light emitting units 11 can directly irradiate the living body with light.

### Effects of Present Example Embodiment

According to the configuration of the present embodiment, the modulation unit 110 dims the one or more light emitting units 11 at a specific frequency, the receiving unit 120 receives biosignals based on light received from a living body by the two or more light receiving units 12, and an adjustment unit 130 adjusts an intensity of the biosignal acquired from each of the light receiving units 12 based on a specific frequency component for the biosignal.

In order to accurately acquire biological information, it is preferable that the intensities of the biosignals (S₁, S₂) are substantially uniform. However, it is difficult to simply normalize the biosignals (S₁, S₂). This is because there is a possibility that both of the biosignals (S₁, S₂) may contain artifact as a body moves (hereinafter referred to as body movement artifact) in addition to pulse. The light receiving units 12 are different from each other in a magnitude of a pulse component in the biosignal depending on how the stick-on type device 10 (10A, 10B, 10C, 10D, 10E) is stuck onto a living body. In addition, the stick-on type device 10 (10A, 10B, 10C, 10D, 10E) is twisted as the body moves. Since the light receiving units 12 are different from each other in a magnitude of noise generated therein depending on how the stick-on type device 10 (10A, 10B, 10C, 10D, 10E) is stuck onto the living body and how the stick-on type device 10 (10A, 10B, 10C, 10D, 10E) is twisted, it is difficult to completely remove only noises such as body movement artifacts from the biosignals (S₁, S₂).

Therefore, in the biological information acquisition device 100, the modulation unit 110 modulates the biosignals (S₁, S₂) at a specific frequency. The specific frequency can be selected from a band that is not affected by pulse components and noises such as body movement artifacts in the biosignals (S₁, S₂). As described above, since the modulation unit 110 of the biological information acquisition device 100 dims the one or more light emitting units 11 at a specific frequency, the biosignals (S₁, S₂) include specific frequency components. The adjustment unit 130 levels the intensities between the biosignals (S₁, S₂) by adjusting the intensities of the biosignals (S₁, S₂) acquired from the individual light receiving units 12 based on the specific frequency components. As a result, the biological information acquisition device 100 can acquire information regarding a living body with high accuracy.

### Second Example Embodiment

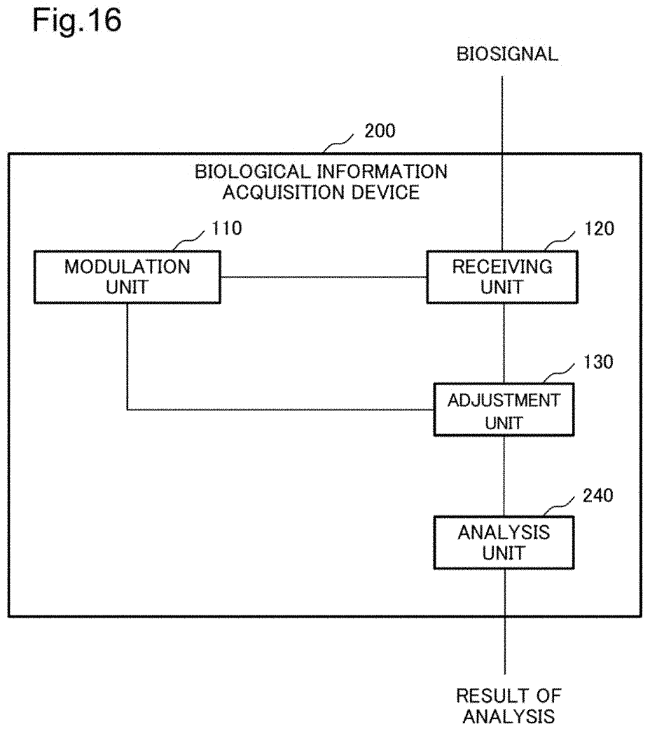

A second example embodiment will be described with reference to FIG. 16. FIG. 16 is a block diagram illustrating a configuration of a biological information acquisition device 200 according to the second example embodiment. As illustrated in FIG. 16, the biological information acquisition device 200 further includes an analysis unit 240 in addition to the modulation unit 110, the receiving unit 120, and the adjustment unit 130. That is, the configuration of the biological information acquisition device 200 according to the second example embodiment is different from the configuration of the biological information acquisition device 100 according to the first example embodiment in that the analysis unit 240 is included.

In the second example embodiment, the components described with the same reference signs in the first example embodiment will not be described.

The analysis unit 240 analyzes a mental or physical condition of a living body based on a biosignal. The analysis unit 240 is an example of an analysis means. In an example, the analysis unit 240 may calculate a light absorption spectrum of a tissue of a living body from a biosignal. Alternatively, the analysis unit 240 may calculate a vibration cycle or a contraction cycle of a part (e.g., a blood vessel) of a living body from a biosignal. Alternatively, the analysis unit 240 calculates at least one index value among an amount, a density, and a concentration (component ratio) of one element (e.g., hemoglobin in blood) of a tissue of a living body from a biosignal. Then, the analysis unit 240 compares the calculated at least one index value with a reference value corresponding to each index value to estimate a mental or physical condition of the living body. For example, when a certain index value is away from the corresponding reference value by a plus or minus a percent or more (α is, for example, a constant having a magnitude of about 5 to 10), the analysis unit 240 determines that the mental or physical condition of the living body indicated by the index value is abnormal. For example, the mental or physical condition of the living body is a condition regarding each of physical health, brain activity (activation), emotion, mental stability, excessive or insufficient nutrition supply, and excessive or insufficient sleep. Specific examples of mental or physical conditions of the living body will be described in the second and third example embodiments.

The analysis unit 240 outputs information indicating the mental or physical condition of the living body as a result of analyzing the biosignal. In an example, the analysis unit 240 displays the information regarding the mental or physical condition of the living body on a screen of a display of a smart device although not illustrated.

### Effects of Present Example Embodiment

According to the configuration of the present embodiment, the modulation unit 110 dims the one or more light emitting units 11 at a specific frequency, the receiving unit 120 receives biosignals based on light received from a living body by the two or more light receiving units 12, and an adjustment unit 130 adjusts an intensity of the biosignal acquired from each of the light receiving units 12 based on a specific frequency component for the biosignal.

As described above, in the biological information acquisition device 200, since the modulation unit 110 dims the one or more light emitting units 11 at the specific frequency, the biosignals include specific frequency components. The adjustment unit 130 levels the intensities between the biosignals by adjusting the intensities of the biosignals acquired from the individual light receiving units 12 based on the specific frequency components. The biological information acquisition device 200 can acquire the information on the living body with high accuracy by selecting a specific frequency from a band that is not affected or hardly affected by noises such as body movement artifacts.

Furthermore, according to the configuration of the present example embodiment, the analysis unit 240 analyzes a mental or physical condition of the living body based on the biosignals. Therefore, the biological information acquisition device 200 can provide a result of analyzing the mental or physical condition of the living body to a person using the biological information acquisition device 200.

### Third Example Embodiment

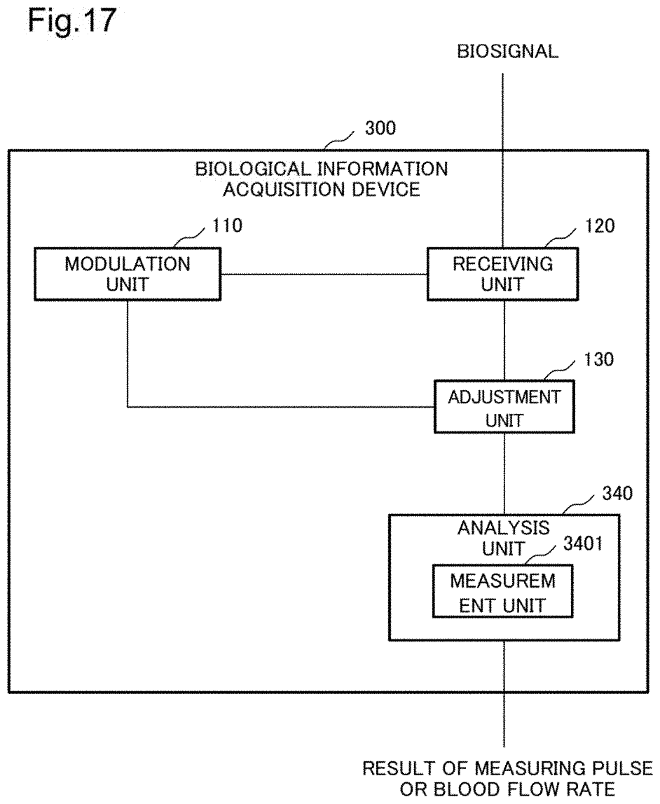

A third example embodiment will be described with reference to FIG. 17. FIG. 17 is a block diagram illustrating a configuration of a biological information acquisition device 300 according to the third example embodiment. As illustrated in FIG. 17, the biological information acquisition device 300 further includes an analysis unit 340 in addition to the modulation unit 110, the receiving unit 120, and the adjustment unit 130. The analysis unit 340 according to the third example embodiment includes a measurement unit 3401.

In the third example embodiment, the components described with the same reference signs in the first or second example embodiment will not be described.

The measurement unit 3401 of the analysis unit 340 measures at least one of a heart rate and a blood flow rate of a living body based on a biosignal. The measurement unit 3401 is an example of a measurement means.

In an example, the measurement unit 3401 calculates a reflection amount of near-infrared light based on a biosignal. The reflection amount of near-infrared light changes when hemoglobin in blood absorbs the near-infrared light (see PTL 2). The measurement unit 3401 measures the heart rate of the living body based on the change in reflection amount of the near-infrared light. The measurement unit 3401 outputs a result of measuring the heart rate of the living body.

Alternatively, it is assumed that hemoglobin content of blood is equal to a reference value. In addition, a thickness of a blood vessel is also assumed. In this case, the measurement unit 3401 may measure a blood flow rate based on a change in reflection amount of near-infrared light (see PTL 2). The measurement unit 3401 outputs a result of measuring the blood flow rate in the living body.

The result of measuring the heart rate of the living body or the blood flow rate of the living body is an example of information indicating a mental or physical condition of the living body. In addition, the result of measuring the heart rate of the living body or the blood flow rate of the living body is also an example of a result of analyzing the living body described in the second example embodiment.

### Effects of Present Example Embodiment

According to the configuration of the present embodiment, the modulation unit 110 dims the one or more light emitting units 11 at a specific frequency, the receiving unit 120 receives biosignals based on light received from a living body by the two or more light receiving units 12, and an adjustment unit 130 adjusts an intensity of the biosignal acquired from each of the light receiving units 12 based on a specific frequency component for the biosignal.

As described above, in the biological information acquisition device 300, since the modulation unit 110 dims the one or more light emitting units 11 at the specific frequency, the biosignals include specific frequency components. The adjustment unit 130 levels the intensities between the biosignals by adjusting the intensities of the biosignals acquired from the individual light receiving units 12 based on the specific frequency components. The biological information acquisition device 300 can acquire the information on the living body with high accuracy by selecting a specific frequency from a band that is not affected or hardly affected by noises such as body movement artifacts.

Furthermore, according to the configuration of the present example embodiment, the analysis unit 340 analyzes a mental or physical condition of the living body based on the biosignals. Therefore, the biological information acquisition device 300 can provide a result of analyzing the mental or physical condition of the living body to a person using the biological information acquisition device 300.

The analysis unit 340 includes a measurement unit 3401 that measures at least one of a heart rate and a blood flow rate of the living body based on the biosignals. Therefore, the biological information acquisition device 300 can provide a result of measuring at least one of the heart rate and the blood flow rate of the living body to a person using the biological information acquisition device 300.

### Fourth Example Embodiment

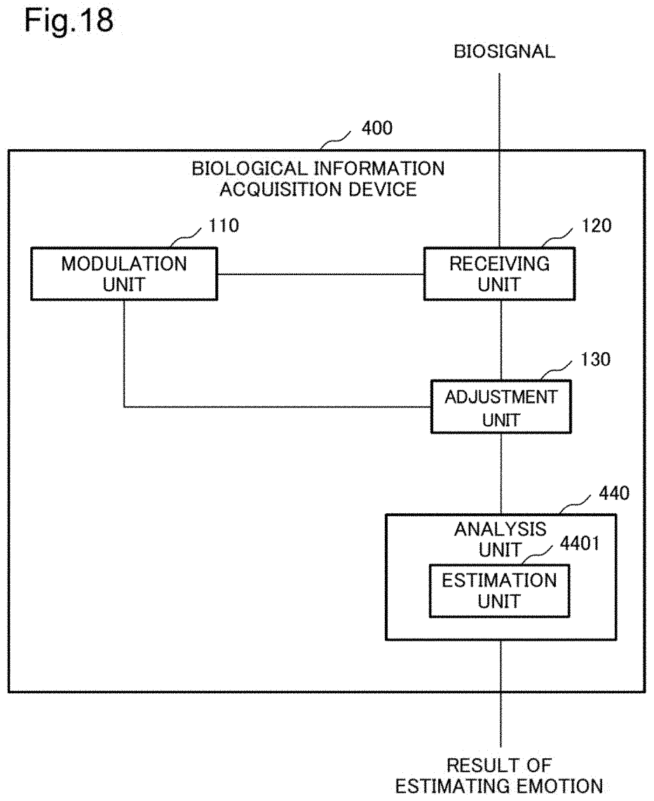

A fourth example embodiment will be described with reference to FIG. 18. FIG. 18 is a block diagram illustrating a configuration of a biological information acquisition device 400 according to the fourth example embodiment. As illustrated in FIG. 18, the biological information acquisition device 400 further includes an analysis unit 440 in addition to the modulation unit 110, the receiving unit 120, and the adjustment unit 130. The configuration of the analysis unit 440 according to the fourth example embodiment is different from the configuration of the analysis unit 240 according to the second example embodiment and the configuration of the analysis unit 340 according to the third example embodiment in that the estimation unit 4401 is included.

In the fourth example embodiment, the components described with the same reference signs in at least one of the first to third example embodiments will not be described.

The estimation unit 4401 estimates an emotion of a living body based on a biosignal. The estimation unit 4401 is an example of an estimation means. In an example, the estimation unit 4401 calculates a reflection amount of near-infrared light based on a biosignal, similarly to the measurement unit 3401 according to the third example embodiment. The estimation unit 4401 measures a heart rate of the living body based on the change in reflection amount of the near-infrared light.

Furthermore, the estimation unit 4401 measures a change in heart rate of the living body. When sympathetic nerves are excited, myocardial contractility is increased and a heart rate is increased in the heart, thereby increasing a cardiac output, and contracting peripheral blood vessels in the whole body. The excitation of the sympathetic nerves is associated with emotions such as excitation, anger, and anxiety. Referring to a table indicating a correspondence relationship of a heart rate and its change with a mental condition (emotion), the estimation unit 4401 estimates various mental conditions (emotions) of the living body based on the change in heart rate of the living body. Then, the estimation unit 4401 outputs a result of estimating the mental condition of the living body.

### Effects of Present Example Embodiment

According to the configuration of the present embodiment, the modulation unit 110 dims the one or more light emitting units 11 at a specific frequency, the receiving unit 120 receives biosignals based on light received from a living body by the two or more light receiving units 12, and an adjustment unit 130 adjusts an intensity of the biosignal acquired from each of the light receiving units 12 based on a specific frequency component for the biosignal.

As described above, in the biological information acquisition device 400, since the modulation unit 110 dims the one or more light emitting units 11 at the specific frequency, the biosignals include specific frequency components. The adjustment unit 130 levels the intensities between the biosignals by adjusting the intensities of the biosignals acquired from the individual light receiving units 12 based on the specific frequency components. The biological information acquisition device 400 can acquire the information on the living body with high accuracy by selecting a specific frequency from a band that is not affected or hardly affected by noises such as body movement artifacts.

Furthermore, according to the configuration of the present example embodiment, the analysis unit 440 analyzes a mental or physical condition of the living body based on the biosignals. Therefore, the biological information acquisition device 400 can provide a result of analyzing the mental or physical condition of the living body to a person using the biological information acquisition device 400.

The analysis unit 440 includes an estimation unit 4401 that estimates an emotion of the living body based on the biosignals. Therefore, the biological information acquisition device 400 can provide a result of analyzing the emotion of the living body to a person using the biological information acquisition device 400.

### Hardware Configuration

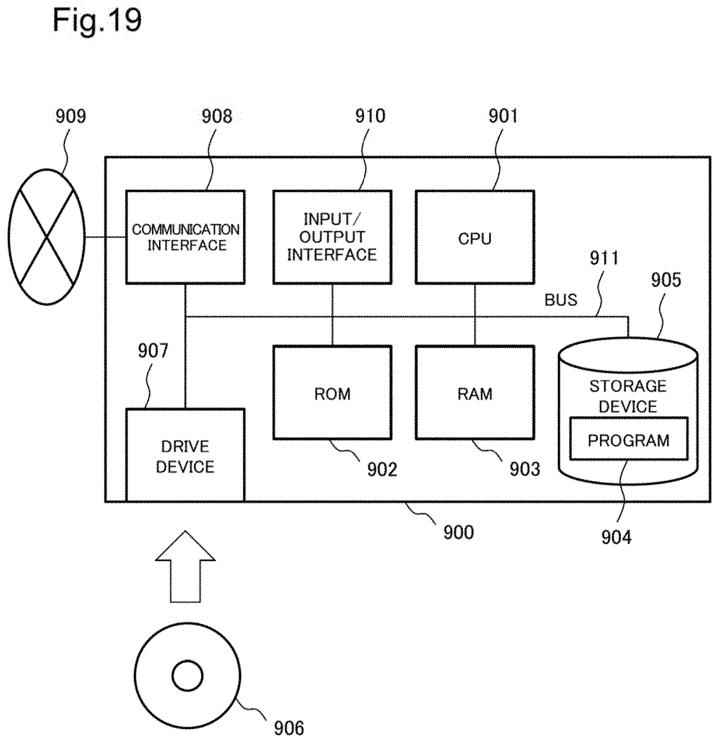

Each of the components of the biological information acquisition devices 100 to 400 described in the first to fourth example embodiments indicates a functional unit block. Some or all of these components are implemented, for example, by an information processing apparatus 900 as illustrated in FIG. 19. FIG. 19 is a block diagram illustrating an example of a hardware configuration of the information processing apparatus 900.

As illustrated in FIG. 19, the information processing apparatus 900 includes the following components as an example.
CPU (Central Processing Unit) 901
ROM (Read Only Memory) 902
RAM (Random Access Memory) 903
Program 904 loaded into the RAM 903
Storage device 905 storing the program 904
Drive device 907 reading and writing a recording medium 906
Communication interface 908 connected to a communication network 909
Input/output interface 910 for inputting/outputting data
Bus 911 connecting the components to each other

The components of the biological information acquisition devices 100 to 400 described in the first to fourth example embodiments are implemented by the CPU 901 reading and executing the program 904 for implementing their functions. The program 904 for implementing the functions of the components is stored, for example, in the storage device 905 or the ROM 902 in advance, and the CPU 901 loads the program 904 into the RAM 903 for execution if necessary. Note that the program 904 may be supplied to the CPU 901 via the communication network 909, or may be stored in advance in the recording medium 906 such that the drive device 907 reads the program to be supplied to the CPU 901.

According to the above-described configuration, each of the biological information acquisition devices 100 to 400 described in the first to fourth example embodiments is implemented as hardware. Therefore, effects similar to those described in the first to fourth example embodiments can be obtained.

### INDUSTRIAL APPLICABILITY

The present invention can be used for a device for acquiring and/or analyzing information regarding a living body, e.g., a heart rate measurement device that measures a heart rate or a blood flow rate of a user, or an emotion estimation device that estimates an emotion of a user.

### REFERENCE SIGNS LIST

100 Biological information acquisition device
110 Modulation unit
120 Receiving unit
130 Adjustment unit
200 Biological information acquisition device
240 Analysis unit
300 Biological information acquisition device
340 Analysis unit
3401 Measurement unit
400 Biological information acquisition device
440 Analysis unit
4401 Estimation unit

What is claimed is:

1. A biological information acquisition device comprising:
a memory configured to store instructions; and
at least one processor configured to execute the instructions to perform:
dimming one or more light emitting elements at a specific frequency;
receiving biosignals based on light received from a living body by two or more optical receiver; and
adjusting an intensity of the biosignal acquired from each of the optical receiver based on a specific frequency component for the biosignal.

2. The biological information acquisition device according to claim 1, wherein
the at least one processor is configured to execute the instructions to perform:
dimming a plurality of light emitting elements that emit light at the same wavelength at the same frequency.

3. The biological information acquisition device according to claim 1, wherein
the at least one processor is configured to execute the instructions to perform:
dimming a plurality of light emitting elements that emit light at different wavelengths at different frequencies.

4. The biological information acquisition device according to claim 1, wherein
the at least one processor is configured to execute the instructions to perform:
dimming a plurality of light emitting elements that emit light at the same wavelength at different frequencies.

5. The biological information acquisition device according to claim 1, wherein
the at least one processor is further configured to execute the instructions to perform:
analyzing a mental or physical condition of the living body based on the biosignals.

6. The biological information acquisition device according to claim 5, wherein
the at least one processor is configured to execute the instructions to perform:
measuring at least one of a heart rate and a blood flow rate of the living body based on the biosignals.

7. The biological information acquisition device according to claim 5, wherein
the at least one processor is configured to execute the instructions to perform:
estimating an emotion of the living body based on the biosignals.

8. A biological information acquisition method comprising:
dimming one or more light emitting elements at a specific frequency;
receiving biosignals based on light received from a living body by two or more optical receiver; and
adjusting an intensity of the biosignal acquired from each of the optical receiver based on a specific frequency component for the biosignal.

9. A non-transitory recording medium storing a program for causing a computer to execute:
dimming one or more light emitting elements at a specific frequency;
receiving biosignals based on light received from a living body by two or more optical receiver; and
adjusting an intensity of the biosignal acquired from each of the optical receiver based on a specific frequency component for the biosignal.
